SEQUENCE LISTING (21 sequences)

SEQ ID NO: 1 - protein, 15 aa, Artificial Sequence
  Chemically Synthesized
XGGGGGGDPGGGGGG

SEQ ID NO: 2 - protein, 8 aa, Artificial Sequence
  Chemically Synthesized
XGGGGGGD

SEQ ID NO: 3 - protein, 7 aa, Artificial Sequence
  Chemically Synthesized
PGGGGGG

SEQ ID NO: 4 - protein, 15 aa, Artificial Sequence
  Chemically Synthesized
XGGGGGGDPGGGGGG

SEQ ID NO: 5 - protein, 8 aa, Artificial Sequence
  Chemically Synthesized
XGGGGGGD

SEQ ID NO: 6 - protein, 7 aa, Artificial Sequence
  Chemically Synthesized
PGGGGGG

SEQ ID NO: 7 - protein, 15 aa, Artificial Sequence
  Chemically Synthesized
XGGGGGGDPGGGGGG

SEQ ID NO: 8 - protein, 8 aa, Artificial Sequence
  Chemically Synthesized
XGGGGGGD

SEQ ID NO: 9 - protein, 7 aa, Artificial Sequence
  Chemically Synthesized
PGGGGGG

SEQ ID NO: 10 - protein, 15 aa, Artificial Sequence
  Chemically Synthesized
XGGGGGGDPGGGGGG

SEQ ID NO: 11 - protein, 8 aa, Artificial Sequence
  Chemically Synthesized
XGGGGGGD

SEQ ID NO: 12 - protein, 7 aa, Artificial Sequence
  Chemically Synthesized
PGGGGGG

SEQ ID NO: 13 - protein, 15 aa, Artificial Sequence
  Chemically Synthesized
XGGGGGGDPGGGGGG

SEQ ID NO: 14 - protein, 8 aa, Artificial Sequence
  Chemically Synthesized
XGGGGGGD

SEQ ID NO: 15 - protein, 7 aa, Artificial Sequence
  Chemically Synthesized
PGGGGGG

SEQ ID NO: 16 - protein, 15 aa, Artificial Sequence
  Artificial Sequence
XGGGGGGDPGGGGGG

SEQ ID NO: 17 - protein, 8 aa, Artificial Sequence
  Chemically Synthesized
XGGGGGGD

SEQ ID NO: 18 - protein, 7 aa, Artificial Sequence
  Chemically Synthesized
PGGGGGG

SEQ ID NO: 19 - protein, 15 aa, Artificial Sequence
  Chemically Synthesized
XGGGGGGDPGGGGGG

SEQ ID NO: 20 - protein, 8 aa, Artificial Sequence
  Chemically Synthesized
XGGGGGGD

SEQ ID NO: 21 - protein, 7 aa, Artificial Sequence
  Chemically Synthesized
PGGGGGG

(19) United States
(12) Patent Application Publication
(10) Pub. No.: US 2009/0002703 A1
(43) Pub. Date: Jan. 1, 2009

(54) METHODS AND SYSTEMS FOR QUANTIFYING ISOBARIC LABELS AND PEPTIDES

(76) Inventor: Craig Edward Parman, Westbrook, CT (US)

Correspondence Address: LOWRIE, LANDO & ANASTASI, LLP, ONE MAIN STREET, SUITE 1100, CAMBRIDGE, MA, 02142, US

(21) Appl. No.: 11/464,930

(22) Filed: Aug. 16, 2006

(51) Int. Cl. G01J 3/28; G01N 33/48

(52) U.S. Cl. 356/326; 702/19

(57) ABSTRACT

Methods and systems for quantifying at least two isobaric labels and/or peptides labeled with different isobaric labels are disclosed. In certain examples, the method comprises fitting a mathematical function to at least two isobaric label fragment peaks in a mass spectrum to quantify simultaneously an amount of at least one peptide present in two different samples.

### FIELD OF THE TECHNOLOGY

Certain examples of the technology described herein are directed to methods and systems for quantifying isobaric labels and peptides. More particularly, in certain embodiments, a method of quantifying two or more isobaric label fragment peaks in a mass spectrum is provided.

### BACKGROUND

Detection of molecules is an important operation in the biological and medical sciences. Such detection often requires the use of specialized label molecules, amplification of a signal, or both, because many molecules of interest are present in low quantities and do not, by themselves, produce detectable signals. Many labels, labeling systems, and signal amplification techniques have been developed. For example, nucleic acid molecules and sequences have been amplified and/or detected using polymerase chain reaction (PCR), ligase chain reaction (LCR), self-sustained sequence replication (3SR), nucleic acid sequence based amplification (NASBA), strand displacement amplification (SDA), and amplification with Qβ replicase (Birkenmeyer and Mushahwar, J. Virological Methods, 35:117-126 (1991); Landegren, Trends Genetics 9:199-202 (1993)). Proteins have been detected using antibody-based detection systems such as sandwich assays (Mailini and Maysef, “A sandwich method for enzyme immunoassay. I. Application to rat and human alpha-fetoprotein” J. Immunol. Methods 8:223-234 (1975)) and enzyme-linked immunosorbent assays (Engvall and Perlmann, “Enzyme-linked immunosorbent assay (ELISA). Quantitative assay of immunoglobulin” Immunochemistry 8:871-874 (1971)), and two-dimensional (2-D) gel electrophoresis (Patton, Biotechniques 28: 944-957 (2000)).

Although these techniques are useful, most have significant drawbacks and limitations. For example, radioactive labels are dangerous and difficult to handle, fluorescent labels have limited capacity for multiplex detection because of limitations on distinguishable labels, and amplification methods can be subject to spurious signal amplification.

### SUMMARY

In accordance with a first aspect, a method of quantifying two or more isobaric labels is disclosed. In certain examples, the method includes applying a mathematical function to at least two isobaric label fragment peaks in a mass spectrum to provide a fitted curve. In some examples, the method may also include determining the relative amount of each isobaric label from the fitted curve. In certain examples, the method may include applying the mathematical function to at least two low mass, isobaric label fragment peaks in the mass spectrum. In yet other examples, the method may include applying the mathematical function to at least two high mass, isobaric label fragment peaks in the mass spectrum. In certain examples, the method may also include applying the mathematical function to all isobaric label fragment peaks in the mass spectrum. In some examples, the method may include determining the intensity of the at least two isobaric label fragment peaks in the mass spectrum from the fitted curve, summing the intensity of the at least two isobaric label fragment peaks in the mass spectrum, and dividing the intensity of at least one isobaric label fragment peak by the summed intensity to determine a relative amount of the at least one isobaric label fragment peak present in a sample. In other examples, the method may include determining an area for each of the at least two isobaric label fragment peaks in the mass spectrum from the fitted curve, summing the areas of the at least two isobaric label fragment peaks in the mass spectrum, and dividing the area of at least one isobaric label fragment peak by the summed areas to determine the relative amount of the at least one isobaric label fragment peak. In yet additional examples, the method may include fitting the mathematical function to each of the at least two isobaric label fragment peaks in the mass spectrum to provide the fitted curve. In some examples, the mathematical function comprises at least one Gaussian function.

In accordance with another aspect, a method of quantifying peptides is provided. In certain examples, the method includes subjecting a sample to fragmentation in a mass spectrometer, the sample comprising a first peptide labeled with a first isobaric label and a second peptide labeled with a second isobaric label, in which fragmentation in the mass spectrometer provides a low mass fragment peak and a high mass fragment peak for each of the first and second isobaric labels. In some examples, the method also further includes applying a mathematical function to at least one of the low mass fragment peaks or the high mass fragment peaks to provide a fitted curve. The method may further include determining the relative amount of each of the first peptide and the second peptide from the fitted curve. In some examples, the first peptide and the second peptide may have the same composition but may originate or be derived from two different samples. In certain examples, the method may include configuring the first isobaric label and the second isobaric label to provide low mass fragments having different mass-to-charge ratios. In other examples, the method may include configuring the mass-to-charge ratios to differ by at least 3 atomic mass units. In some examples, each low mass fragment peak and each high mass fragment peak has an intensity, and the determining step may further comprise determining the intensity of the peaks of the fitted curve, summing the intensity of the peaks of the fitted curve, dividing the intensity of at least one of the fitted curve peaks by the summed intensity to determine a relative amount of at least one isobaric label fragment peak, and correlating the determined relative amount of the at least one isobaric label fragment peak with a relative amount of a peptide. In some examples, the first peptide and the second peptide may have the same composition but are derived from at least two different samples. In other examples, the first peptide and the second peptide may have different compositions or sequences.

In accordance with an additional aspect, a mass spectrometer is disclosed. In certain examples, the mass spectrometer comprises a first stage configured to select at least one peptide originating from two different samples that have been labeled with two different isobaric labels, and a second stage in fluid communication with the first stage, the second stage configured to fragment the two, different isobaric labels to provide two low mass fragments and two high mass fragments. In some examples, the mass spectrometer may further include a processing device electrically coupled to the second stage, the processing device configured to quantify simultaneously the two or more different fragmented isobaric labels. In certain examples, each of the first stage and the second stage may be configured as a quadrupole mass spectrometer. In other examples, the first stage may be configured to pass two or more peptides with a mass-to-charge ratio within a selected mass-to-charge window. In yet additional examples, the processing device may include an algorithm that is configured to quantify the peptides by fitting a mathematical function to the two low mass fragments to quantify simultaneously a relative amount of the at least two peptides.

In accordance with another aspect, a computer readable medium comprising an algorithm for simultaneously quantifying at least two different fragmented isobaric labels, the algorithm configured to fit a mathematical function to at least two isobaric label fragment peaks in a mass spectrum to quantify simultaneously a relative amount of the at least two peptides is provided. In certain examples, the two peptides may have the same composition but be derived from different samples, whereas in other examples the peptides may have a different composition and be derived from the same or different samples.

In accordance with an additional aspect, a kit is disclosed. In certain examples, the kit includes an isobaric label, a computer readable medium comprising an algorithm configured to quantify simultaneously two or more species, e.g., a single peptide derived from at least two different samples, and instructions for using the isobaric label and the algorithm to quantify peptides in a sample. In some examples, the kit may include two or more isobaric labels. In certain examples, the kit may include two or more isobaric labels. In additional examples, the algorithm may be configured to quantify the at least two peptides by fitting a mathematical function to two low mass fragments from isobaric labels to quantify simultaneously a relative amount of the at least two peptides

In accordance with another aspect, a method of quantifying at least two peptides labeled with different isobaric labels, the method comprising fitting a mathematical function to two isobaric label fragment peaks in a mass spectrum to quantify simultaneously a relative amount of the at least two peptides present in a sample is provided, e.g., to quantify the relative amount of at least one peptide derived from at least two different samples.

It will be recognized by the person of ordinary skill in the art, given the benefit of this disclosure, that certain embodiments of the methods, devices and systems disclosed herein can reduce the time required to quantify various analytes. Other features, aspects and advantages of the technology disclosed herein are discussed in detail below.

### BRIEF DESCRIPTION OF THE FIGURES

Certain examples are described below with reference to the accompanying figures in which:

FIG. 1 is a schematic of using tandem MS/MS with the methods and systems disclosed herein, in accordance with certain examples;

FIG. 2 is a diagram of two isobaric labels and their produced fragments, in accordance with certain examples;

FIG. 3 is a mass spectrum showing two isobaric label fragment peaks, in accordance with certain examples;

FIG. 4 is a histogram of several spectra showing the distribution of mass peaks for two isobaric label fragment peaks and a fitting curve used to fit the two isobaric label fragment peaks, in accordance with certain examples;

FIG. 5 is a flow-chart showing an illustrative process that may be used with the methods and systems disclosed herein, in accordance with certain examples;

FIG. 6 is a flow-chart showing an illustrative process for quantifying peptides and identifying the sequence of the peptides, in accordance with certain examples;

FIG. 7 is a flow-chart showing an illustrative process to correlate predicted peptide sequences with a list of quantified peptides to provide a list of quantified peptides, in accordance with certain examples;

FIG. 8 is an example of a general-purpose computer system, in accordance with certain examples;

FIG. 9 is an example of a storage system, in accordance with certain examples;

FIG. 10 is an example of a device for mass spectroscopy, in accordance with certain examples;

FIG. 11 is a mass spectrum showing a selected mass-to-charge window, in accordance with certain examples;

FIG. 12 is a mass spectrum showing a selected mass-to-charge window and peak intensities, in accordance with certain examples;

FIG. 13 is a mass spectrum showing a selected mass-to-charge window and showing mass peaks as line with circles at the top, in accordance with certain examples;

FIG. 14 is a fitting curve showing a best fit for a selected peak in a mass spectrum, in accordance with certain examples;

FIG. 15 is a mass spectrum showing shaded regions that may be used to extract the intensity for each isobaric label fragment peak in the mass spectrum, in accordance with certain examples;

FIG. 16 is a series of graphs showing a comparison between experiments that picked seven isobaric label fragment peaks and experiments that picked four isobaric label fragment peaks, in accordance with certain examples; and

FIG. 17 is a series of graphs showing a comparison between experiments that picked seven isobaric label fragment peaks and an experiment that picked two isobaric label fragment peaks, in accordance with certain examples.

It will be recognized by the person of ordinary skill in the art, given the benefit of this disclosure, that certain features in the figures may have been enlarged, reduced, distorted or otherwise drawn in a non-conventional manner to facilitate a better understanding of the new and useful technology described herein.

### DETAILED DESCRIPTION

The methods and devices disclosed herein will be recognized by the person of ordinary skill in the art, given the benefit of this disclosure, to provide a significant advance in analyzing mass spectra. In certain embodiments, by applying a mathematical function to all analyte peaks in a selected window of a mass spectrum, throughput and accuracy may be increased, sample processing time may be reduced and usability may be enhanced.

As used herein, the term “at least two peptides” refers to at least two peptides being present in a sample. The composition of the at least two peptides may be substantially the same, or exactly the same, but the peptides present may originate or be derived from at least two different samples, e.g., lactate dehydrogenase from two different patient samples that have been pooled. The peptides from different samples may be separated from other species in the samples, labeled and analyzed using the methods disclosed herein. In other examples, the at least two peptides may have different compositions, e.g., may be chemically different, and may originate or be derived from the same or different samples.

In accordance with certain examples, the methods disclosed herein may be used to simultaneously quantify two or more peaks in a mass spectrum. In certain examples, the two or more peaks in the mass spectrum represent two or more species that have been labeled with a reporter signal each of which has been fragmented to provide peaks having a different mass-to-charge ratio. In some examples, a plurality of peaks that represent species having a fragmented reporter signal may be simultaneously quantified. In certain examples, the species may be quantified in relative amounts, e.g., the percentage of each species present in a sample, whereas in other examples, the peaks may be quantified in absolute amounts, e.g., the mass or concentration of each species in the sample.

In accordance with certain examples, two or more reporter signals, such as those described in commonly owned U.S. Pat. No. 6,824,981, the entire disclosure of which is hereby incorporated herein by reference for all purposes, may be used with the methods disclosed herein. For convenience purposes only and without limitation, the term reporter signal is used interchangeably herein in some instances with the term “isobaric label.” In brief, two or more species may be labeled with two or more different, isobaric labels that are susceptible to fragmentation in a mass spectrometer. For example, different isobaric labels may be added to a sample having two or more analytes. In some examples, a sample may include a single type of analyte that is derived from two or more samples, e.g., a spot from a 2-D gel that separated proteins from 2 or more samples. The labeled analytes may be selected from other components in the sample using a first mass selector. The isobaric labels may then be fragmented in a second stage using a collision cell or other fragmentation device. Before fragmentation, the isobaric labels have the same mass-to-charge ratio. After fragmentation, the isobaric labels are selected such that the isobaric label fragments from different labels have different mass-to-charge ratios. For example, the following two labels may be used to provide isobaric label fragments having different mass-to-charge ratios.

X-GGGGGGDPGGGGGG
X-GGGGGGDPGGGGGG

The underlining shown in the labels above indicates an amino acid residue having a heavy isotope, e.g., ²H, ³H, ¹³C, ¹⁵N, etc. In some examples, an amino acid containing an isotopically heavy nitrogen (¹⁵N) and two isotopically heavy carbon atoms (¹³C) may be used to provide a 3 Dalton shift. The X residue refers to the remainder of the analyte to which the label has been linked. The above labels, and at least some of those disclosed in U.S. Pat. No. 6,824,981 are designed to fragment between the aspartate and proline residues. Subsequent to fragmentation, the following fragments may result.

X-GGGGGGD + PGGGGGG
X-GGGGGGD + PGGGGGG

The second proline fragment (PGGGGGG) has a mass-to-charge ratio that is higher than the first proline fragment (PGGGGGG) due to the presence of the isotopically heavy amino acid. Any number of isobaric labels that provide fragments having different mass-to-charge ratios may be used in the methods disclosed herein. Additionally, the position of the fragmentable aspartate-proline (DP) bond may be altered with respect to the other amino acid residues in the isobaric label in order to achieve the mass differences in the fragments. In this case, isotopically heavy amino acids may or may not be employed in the isobaric label.

In accordance with certain examples, by selecting isobaric labels that provide fragments having close, but not the same, mass-to-charge ratios, the amount of each analyte present in a sample may be quantified by fitting of the peaks representative of the isobaric label fragments. For example, a single analyte from different samples may be labeled with different isobaric labels. In a typical example, either before labeling, after labeling or both, the analytes may be subjected to separation and/or analysis to determine the relative sizes of the labeled analytes. The labeled analytes may then be injected into a tandem mass spectrometer for analysis. For example, the labeled materials may be combined and fractionated by sodium dodecyl sulfate polyacrylamide gel electrophoresis. One or more regions of the polyacrylamide gel with relative molecular weight 65 kDa may be excised with a razor, and the gel piece macerated and subjected to trypsin digestion. The resulting peptides may be desalted and concentrated using chromatography, e.g., high performance liquid chromatography, with a reverse-phase C18 column and the purified peptides may be subjected to tandem mass spectrometry.

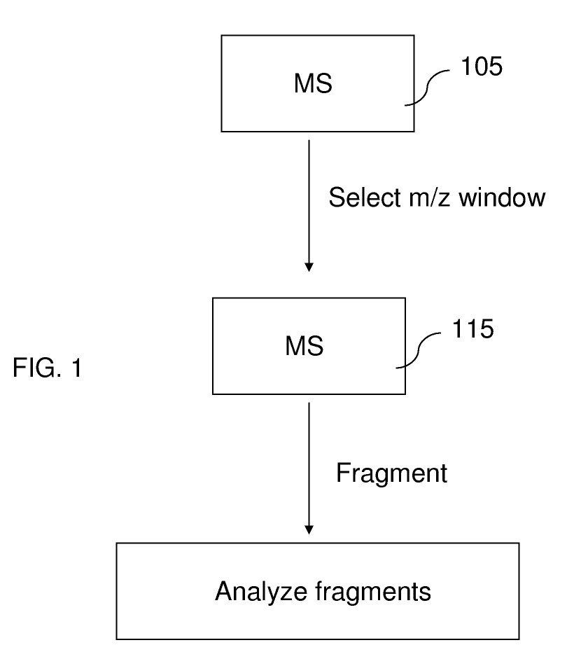

In certain examples, the first stage of the mass spectrometer may be configured to select labeled analytes having a desired mass-to-charge ratio, which reduces the number of labeled analytes that are being analyzed or passed to the second stage. Labeled analytes having a selected mass-to-charge ratio may then be passed to a second stage where the labeled analytes may be fragmented in a collision cell or the like. Fragmentation of labeled analytes causes the different isobaric labels to break at a desired site, e.g., between an aspartate residue and a proline residue. Fragmentation of the isobaric labels typically provides a low mass signal and a high mass signal. For example and referring to FIG. 1, a first stage 105 can select labeled analytes having a similar mass-to-charge ratio or a mass-to-charge ratio within a selected mass window. A second stage 115 may fragment the isobaric mass label of the labeled peptide 110 to provide a low mass fragment and a high mass fragment. As used herein, the low mass fragment is, as the name suggests, the fragment having the lower mass. In certain embodiments, the low mass fragment is the fragment that includes a proline residue at its amino terminus with the high mass fragment including an aspartate at its carboxy terminus. Either the signal from the low mass fragment, the signal from the high mass fragment or both may be used with the methods disclosed herein to quantify the species present in a sample.

In accordance with certain examples, the methods disclosed herein may be used to quantify numerous different types of analytes including biological and non-biological analytes. In certain instances, non-biological analytes may include organic compositions, inorganic compositions and other compositions not typically found in a biological system. Biological analytes include, but are not limited to, proteins, peptides, carbohydrates, monosaccharides, disaccharides, fatty acids, lipids, nucleic acids, second messengers or other species commonly found in a biological system. Any analyte to which a reporter signal may be attached may be quantified using the methods disclosed herein. It will be within the ability of the person of ordinary skill in the art, given the benefit of this disclosure, to select suitable analytes for use with the methods disclosed herein.

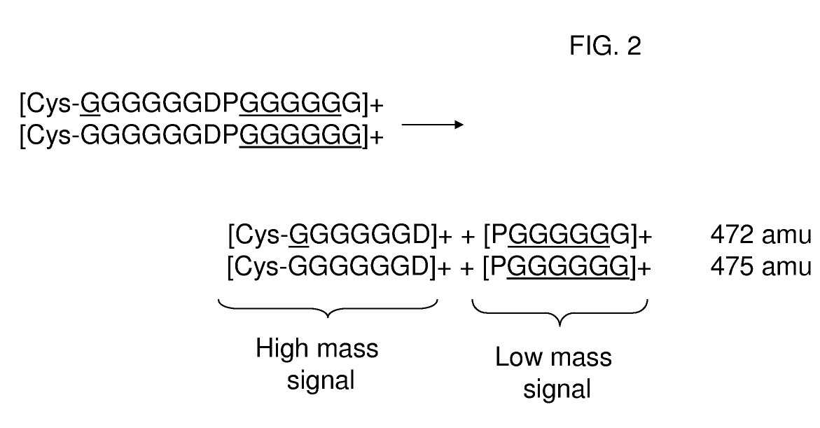

In accordance with certain examples, by varying the isotopic content of the isobaric labels, the mass-to-charge ratio of the high mass fragment and the low mass fragment of different labels may be different. This feature may be used to analyze peptides resulting from digestion of a protein sample. In a simple illustration, a first complex peptide sample, e.g., peptides from an enzymatic digestion of a serum sample, may be labeled with a first isobaric label. A second complex peptide sample, e.g., peptides from an enzymatic digestion of a plasma sample may be labeled with a second isobaric label. The first and second labeled peptide mixtures may be injected into a first stage to select out the labeled peptides of interest from the remainder of the mixture. The selected, labeled first and second peptides may then be passed to a fragmentation stage such that fragmentation of the isobaric labels occurs and the relative abundances of the peptide or peptides of interest between the serum and plasma sample may be determined based upon the labeled peptide constituents. For example, the two isobaric labels shown in FIG. 2 are used to illustrate this embodiment. The underlining shown in FIG. 2 indicates a residue having a heavy isotope. In certain examples, an underlined residue has a mass that is three atomic mass units larger than a non-heavy isotopic residue. For example, an underlined G refers to a glycine that has a mass that is three atomic mass units larger than a non-underlined glycine. For the labeled peptides shown in FIG. 2, the Cys group represents a cysteine side chain of a peptide to which the label has been attached. In the first stage where the peptide mass-to-charge is selected, suitable mass spectrometer parameters may be selected such that both peptides are passed to a second stage. At the second stage, the isobaric label fragments between the aspartate residue (D) and the proline residue (P). Fragmentation of the first label provides a low mass signal having an atomic mass of about 472 amu. Fragmentation of the second label provides a low mass signal having an atomic mass of about 475 amu. Fragmentation also provides a high mass signal for each of the peptides which, as discussed in more detail below, may be used to determine the sequence of the peptides. Either the low mass signal or the high mass signal or both may be used in the quantitation methods described herein.

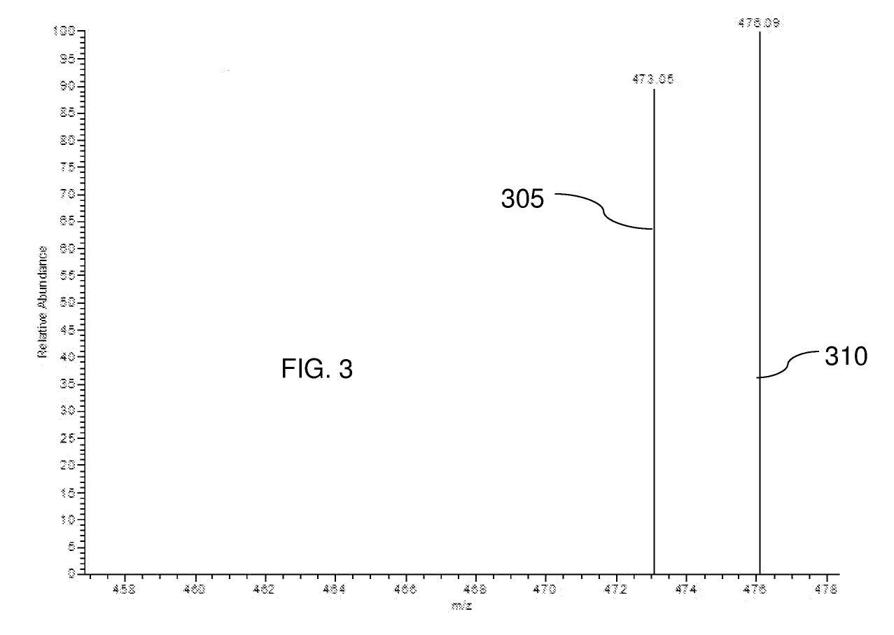

In accordance with certain embodiments, a simplified mass spectrum showing a low mass signal that has resulted from fragmentation of a first and second isobaric label is shown in FIG. 3. Depending on selection of the particular label, the amount of mass separation of the isobaric label may be controlled. In the simple example shown in FIGS. 2 and 3, the isobaric labels were selected such that the low mass fragments would be separated by 3 atomic mass units (amu's). The first isobaric label fragment 305 has a mass-to-charge (m/z) of about 473 and the second isobaric label fragment 310 has an m/z of about 476. In certain examples, the isobaric labels may be separated by at least about 3 amu's or more. While the methods disclosed herein may be used with isobaric label fragments that are separated by 1 amu or 2 amu's, intrinsic isotope content within a species may reduce the overall accuracy. By increasing the mass separation of the isotopic label fragments to 3 amu's or more, the background signal, and any intrinsic isotope effects, may be reduced to provide more accurate spectra for use in the methods disclosed herein.

In certain embodiments, the relative amount of isobaric label fragments 305 and 310 may be determined simultaneously by applying a mathematical function to the two isobaric label fragments 305 and 310 to provide a fitted curve or fitting function. The fitting function provides a best fit, curve of all the peaks representing low mass signals, high mass signals or both from the isobaric labels. The areas under each curve, the intensity of each curve or other selected parameter may be used to determine the relative amounts of each isobaric label fragment present. The relative amount of any isobaric label fragment is directly proportional to the relative amount of the particular species that was labeled with that isobaric label. In certain examples, a fitting function may be applied to all of the peaks from the low mass signals (or high mass signals as the case may be) that result subsequent to fragmentation. By fitting a curve to all of the peaks at once, the relative amounts of all isobaric labels, and the relative amounts of the analytes, may be determined simultaneously.

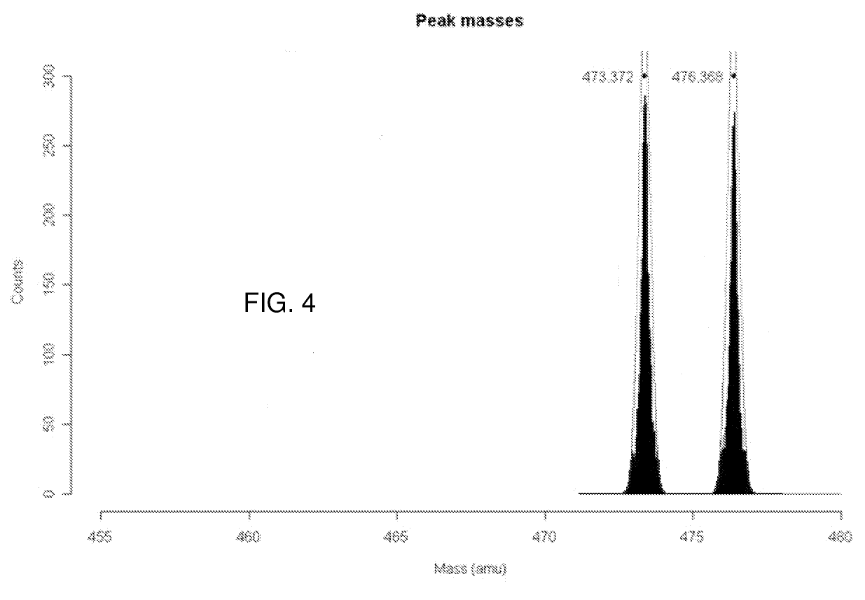

In accordance with certain examples, the exact type of mathematical function used to provide a fitted curve may vary to best describe the distribution of mass peaks observed for a particular configuration. In certain examples, the distribution of the measured mass of a species entering into the second stage may be expected to be Gaussian, and Gaussian functions, e.g., a plurality of summed Gaussian functions, may be used to provide a best overall fit for all of the low mass signal peaks (or the high mass signal peaks as the case may be). Referring now to FIG. 4, a curve based on the sum of two individual Gaussian functions may be applied to two peaks to provide the relative amounts of each isobaric label fragment present in the sample. As discussed in more detail below, the mean in each Gaussian function is the expected mass and the standard deviation in each Gaussian function is the peak width. Unlike other methods of fitting mass spectra, which sequentially fit each peak to try and identify the amounts of each species in a sample, the methods disclosed herein apply a mathematical function to obtain an overall fit for all of the peaks simultaneously, e.g., a global best fit for all the peaks in the spectrum. By applying the mathematical function to all peaks simultaneously, the fit of any particular peak may be different than if the mathematical function is only applied to that particular peak, but the overall, global fit for all of the peaks may be a best fit. It will be recognized by the person of ordinary skill in the art, given the benefit of this disclosure, that even though the peaks may be fit simultaneously to quantify label fragments (or peptides as the case may be), the results of such quantitation need not be displayed, printed or outputted at the same time.

In the simplified example shown in FIG. 4, Gaussian functions have been applied to the distribution of the two mass peaks resulting from fragmentation of the isobaric labels. Using the fitted Gaussian functions, the relative amount of each isobaric label fragment may be calculated in many different manners, e.g., using the area under each curve, peak intensity, etc. For example, the area under any one curve may be divided by the summed area to provide a relative amount for each isobaric label fragment. Alternatively, the intensities of each peak may be summed, and the intensity of any particular peak may be divided by the summed intensity to provide a relative amount for each isobaric label fragment. In certain embodiments, both the area under the curve and the peak intensity may be used. The methods just described provide the relative amounts, e.g., percentages or fraction, of each isobaric label present in a sample. In many instances, determination of the relative amounts of the different peptides present in a sample is sufficient to provide useful information, and therefore, it is unnecessary to determine the absolute amount or concentration of each peptide present.

In accordance with certain embodiments, the absolute amount of each isobaric label present in a sample may be determined. Determination of absolute amounts may be useful, for example, where the isobaric labels are used in a clinical or diagnostic setting. In certain examples, the absolute amount of each isobaric label may be determined by comparison of the peak intensity with a calibration curve. In other examples, the absolute amount of each isobaric label may be determined by measuring a signal increase in the absence and presence of sample. For example, an internal standard may be included that provides a signal at each mass-to-charge ratio for a set of isobaric labels. Upon addition of sample to the internal standard, the percentage or fold increase in the signal may be used to assess how much of each isobaric label is present in the sample. Other methods of determining absolute amounts of each isobaric label will be readily selected by the person of ordinary skill in the art, given the benefit of this disclosure.

In accordance with certain examples, a significant time savings may be achieved by fitting all of the peaks simultaneously, particularly when a plurality of isobaric labels are used to label a sample. For example, it may be possible to analyze a sample having 100 or more different analytes, each labeled with a different isobaric label. The resulting signal, after fragmentation of the isobaric labels, would include 100 or more peaks that represent the various isobaric labels in the sample. By simultaneously fitting all of the 100 or more peaks, the relative amounts of each analyte in the sample may be rapidly determined.

In accordance with certain examples, various mathematical functions may be used to fit the low mass signal peaks or the high mass signal peaks or both. A function configured to fit a curve to a Gaussian distribution, a Laplacian distribution, a Maxwellian distribution, a Rayleigh distribution, a Uniform distribution, a Lorentzian distribution (Cauchy or Breit-Wigner), hybrid Gaussian-Cauchy distribution, empirically transformed Gaussian distribution, parabolic Lorentzian-modified Gaussian distribution, or Haarhoff-van der Linde analysis, models commonly used to characterize skewed peaks, sum of square roots peak models, combinations of leading and trailing edge functions or other selected distribution or models may be used to provide a best fit to the various peaks in a selected mass-to-charge window. In certain examples, a peak within a selected mass-to-charge window may be fit using a Gaussian function. An example of a Gaussian probability density function is shown in the following equation where μ is the mean (the expected mass) and σ is the standard deviation (the expected width of the peak).

$$f ( x ; μ , σ ) = \frac{1}{σ 2 π} exp ( - \frac{{( x - μ )}^{2}}{2 {σ}^{2}} )$$

In examples where a plurality of peaks are present, each peak may be fit using, for example, a function such as the probability function shown above, with the overall function for the entire curve being a sum of the individual functions. Suitable algorithms for implementing a function to fit to such distributions are well known to those skilled in the art. Additional algorithms for fitting a function to a selected distribution will be readily selected by the person of ordinary skill in the art, given the benefit of this disclosure. In some examples, the overall function may be the sum of two different functions. For example, it may be desirable to fit a first peak using a Gaussian function and fit a second peak using a Laplacian function with the overall function being the sum of the Gaussian function and the Laplacian function.

In accordance with certain examples, the accuracy of the curve fitted to the peaks may be assessed using conventional error analysis functions. For example, a least squares analysis, Chi-square analysis, Poisson statistical error, or other suitable statistical methods of assessing error may be used to evaluate the fit. In certain examples, the goodness of fit may be determined, for example, by the total signal found by the quantification process. The fit that yields the largest signal may be considered the best fit. Additional methods of assessing the goodness of the fit of the peaks will be readily selected by the person of ordinary skill in the art, given the benefit of this disclosure.

In accordance with certain examples, the fitting function may be applied in an iterative manner, and error after each iteration may be determined prior to the next iteration. In certain examples, when the error is minimized, a best fit function should be provided. The exact iterative method used may vary, and in certain examples, the iterative method used depends on the exact distribution model used for the fitting process. It will be within the ability of the person of ordinary skill in the art, given the benefit of this disclosure, to select suitable iterative methods.

In accordance with certain examples, once the best fit for all of the peaks has been identified, several different methods may be used to ascertain the relative amounts of each label present in the sample. In certain examples, the areas under each curve of the fitted curve may be summed. Summing of the curves may be performed manually or may be automated using a computer or other processing device. The area of any particular curve may be divided by the summed areas to obtain a relative amount, e.g., a percentage, for an isobaric label fragment represented by that particular curve. For example, in the case where two low mass, isobaric label fragments are used, a first low mass fragment may provide an area of 300 and a second low mass fragment mass provide an area of 700. By summing the two areas and dividing the area of each peak by the total area, the first low mass fragment is present at a relative amount of 30% in the sample, and the second low mass fragment is present at a relative amount of 70% in the sample. Correspondingly, the analyte to which the first low mass fragment is bound to is present at a relative amount of 30% in the sample, and the analyte to which the second low mass fragment is bound to is present at a relative amount of 70% in the sample. It will be within the ability of the person of ordinary skill in the art, given the benefit of this disclosure, to use peak areas to quantify the amount of an analyte present in a sample.

In accordance with certain examples, the intensity of each peak of the fitted curve may be used to determine the relative amount of each analyte present in a sample. For example, the intensities of each peak may be summed. Summing of the intensities may be performed manually or may be automated using a computer or other processing device. The intensity of any particular curve may be divided by the summed intensities to obtain a relative amount, e.g., a percentage, for an isobaric label fragment represented by that particular curve. For example, in the case where two low mass, isobaric label fragments are used, a first low mass fragment may provide an intensity of 70 and a second low mass fragment may provide an intensity of 100. By summing the two intensities and dividing the intensity of each peak by the summed intensities, the first low mass fragment is present at a relative amount of 70/170=41.2% in the sample, and the second low mass fragment is present at a relative amount of 100/170=58.8% in the sample. Correspondingly, the analyte to which the first low mass fragment is bound to is present at a relative amount of 41.2% in the sample, and the analyte to which the second low mass fragment is bound to is present at a relative amount of 58.8% in the sample. It will be within the ability of the person of ordinary skill in the art, given the benefit of this disclosure, to use peak intensities to quantify the amount of an analyte present in a sample.

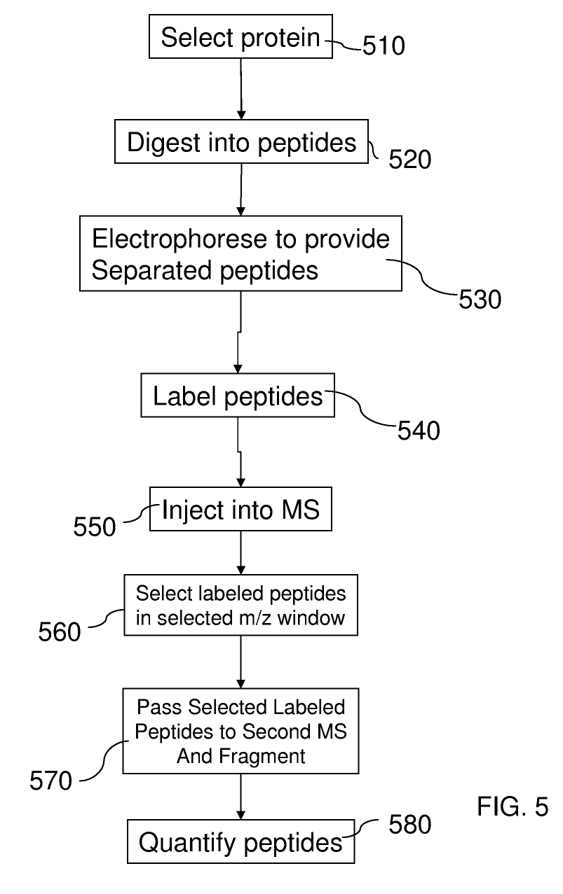

In accordance with certain examples, the methods disclosed herein may be used in conjunction with known methods to identify a sequence of an unknown protein. Referring to FIG. 5, a flow chart showing this process is shown. In a first step, a protein is selected 510. Because proteins are typically too large to be analyzed directly in a mass spectrometer, the protein may be digested into peptides 520 to break the protein into smaller fragments. To determine the size of the peptides, the peptides may be subjected to an electrophoresis step 530 to provide separated peptides. The size of the separated peptides may be determined using known standards, e.g., SDS-PAGE standards. In some embodiments, the peptides may be labeled prior to separation, whereas in other embodiments, the peptides are separated to provide the user some indication of the size of the various peptides so that the user can select a desired mass-to-charge window for the first MS stage in a tandem mass spectrometer, and the peptides may be labeled subsequent to separation. In the case where peptides are separated prior to labeling, the peptide fragments may be subjected to a labeling step 540 to provide labeled peptide fragments. The labeled peptide fragments may then be injected 550 into a first MS device. In the first MS device, the user may select 560 a desired mass-to-charge window, which is typically selected based on the size of the peptides as determined from the electrophoresis step. The first MS device passes 570 labeled peptides having a mass-to-charge ratio within the selected window to the second MS device. The second MS device 570 is configured to fragment the isobaric labels of the passed, labeled peptides into low and high mass fragments which may be used to quantify the peptides, as described herein, using the resulting peaks and the result of the fitting of the resulting peaks. The process just described may be repeated by selecting a different mass-to-charge window and repeating the process until all of the peptides resulting from protein digestion have been quantified.

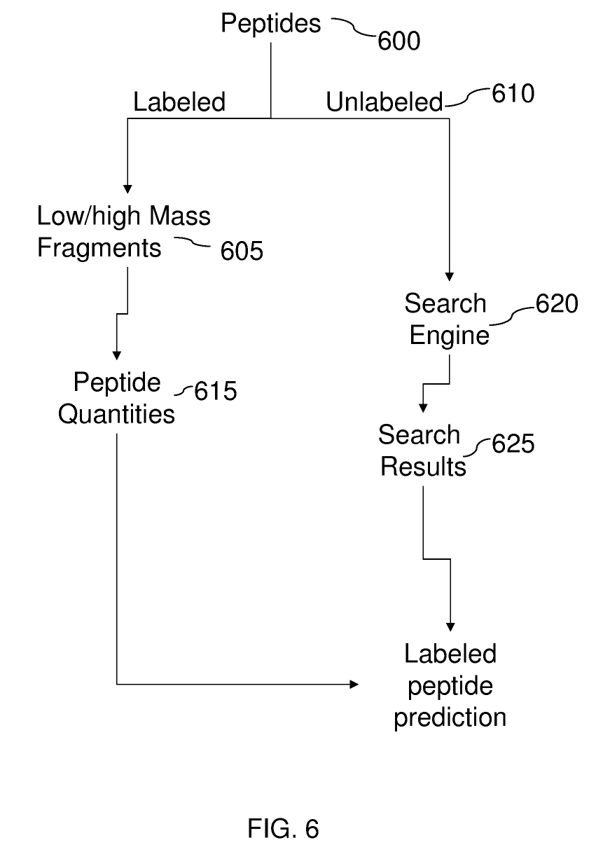

In accordance with certain examples, the method described in FIG. 5 provides the relative amount of peptides present in a sample but does not account for the identify of each of the peptides. To sequence a protein, it may be desirable to know both the amount of peptide fragments that result from digestion as well as the composition of such peptide fragments. To determine the sequence of the peptide fragments, conventional protein sequencing tools and methods may be used. For example, the unlabeled peptide fragments that result from fragmentation may be used to determine the sequence of the peptide. This process may be performed simultaneously with quantitation where low or high mass fragments may be used to quantitate, and unlabeled fragments may be used to predict the sequence of the peptide. This process is illustrated schematically in FIG. 6. Fragments 600 produced from a second MS stage may be grouped as low/high mass fragments 605, which may be used with the methods disclosed herein to provide quantitative amounts of each of the peptide fragments. Unlabeled fragments 610 may be submitted to or used in an existing search engine 620 to provide potential sequences for each of the peptide fragments. Illustrative search engines include, but are not limited to, MASCOT (Matrix Science (Boston, Mass.)), SEQUEST (Scripps Research Institute), and X!TANDEM (The Global Proteome Machine Organization). Without wishing to be bound by any particular scientific theory, the search engines may query one or more databases to attempt to match mass spectra patterns of known peptide sequences with peaks from unknown peptide sequences. The output of the search engine will typically be a search result 625 that is a list of potential sequences rather than a single sequence. The search results 625 may be combined with the quantitation results 615 in a list 630 for examination by a user. In certain embodiments, one or more additional programs or databases may be used along with the protein identification and quantitation list 630 to provide potential sequences of the original protein 640.

In accordance with certain examples, if a user already knows the identity of a protein prior to analysis, then a user may simply enter the protein sequence and/or resulting peptide sequences such that the output of the list 630 may include a peptide sequence list and a relative amount of peptide present for each peptide sequence in the list. In instances where the protein sequence is known, digestion of the protein with a particular enzyme, e.g., trypsin, will provide protein fragments whose sequences can be easily predicted based on the known enzymatic action of a selected enzyme, e.g., trypsin cleaves C-terminal to arginine and lysine residues. Such analysis may be useful in the clinical setting where the levels of a particular protein may be monitored to determine if protein expression increases or decreases in response to a stimulus such as a drug.

In examples where both a protein sequence and the amount of a particular peptide sequence are desirable, the amount of each label quantified may be correlated with a particular peptide sequence. This process may be automated by entering the particular label used with a particular peptide fragment. For example, as the total mass of any particular peptide fragment may be determined using gel electrophoresis, a user may enter the sequence of a label used with a particular peptide fragment size. Using the low and high mass fragments, the amount of label, as determined from the low mass fragments, may be correlated to a particular peptide sequence, as determined using unlabeled mass fragments, such that the amount and identity for any particular peptide fragment may be determined using the methods disclosed herein.

In accordance with certain examples, the methods disclosed herein may be used to identify sequences of a plurality of proteins at the same time. Different isobaric labels may be used to label peptides resulting from digestion of multiple biological samples containing a plurality of different proteins. By proper selection of the isobaric labels, labeled peptide fragments resulting from digestion of different protein samples may be quantified from a single mass spectrum. For example, for a first set of complex protein samples, e.g. control patient serum samples, a set of isobaric labels that provide low mass fragments within a mass-to-charge window ranging from about 450-500 may be used. A set of isobaric labels that provide low mass fragments low mass fragments within a mass-to-charge window ranging from about 500-550 may be used to analyze a second set of complex protein samples, e.g. stage 1 through IV ovarian cancer patient serum samples. The two sets of serum samples may be combined, depleted of high abundance proteins (e.g. albumin, immunoglobulin G) using a commercially-available affinity depletion column, proteolytically digested with trypsin, and subjected to high performance liquid chromatography combined with tandem mass spectrometry. By varying the mass-to-charge ratio window where a set of labeled peptides is expected to provide a peak in the first stage of the mass spectrometer, a plurality of different proteins may be quantified and analyzed simultaneously using a plurality of different isobaric labels. Candidate protein biomarkers may be identified based upon reproducible abundance differences determined between the control and diseased serum samples.

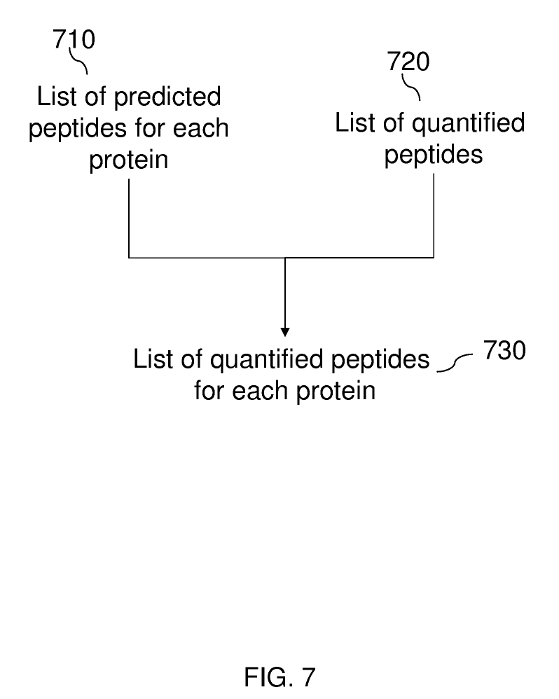

In accordance with certain examples, for each protein analyzed using the methods disclosed herein, a list of quantified peptides may be provided. For example and referring to FIG. 7, a list of predicted peptides for each protein 710, which may be the list provided at 625 in FIG. 6, may be correlated with a list of quantified peptides 720, which may be the list 615 provided in FIG. 6, to provide a list 730 of quantified peptides for each protein subjected to analysis. For a typical end user, the list 730 may be all that is outputted to a screen or printing device. In a clinical setting, the list 730 would allow a user to determine if a particular peptide is present at an amount above or below a threshold range, which may be indicative of a disease state or condition. Additional uses of the methods disclosed herein are discussed in more detail below.

In accordance with certain examples, the methods disclosed herein may be used to quantify peptides from numerous different samples. For example, since each peptide may contain a label that provides a unique signature to the peptide, peptides from different sources, e.g., different body fluids, may be combined together prior to analysis. The ability to analyze a sample made up of different fluids provides for rapid analysis of multiple, different analytes.

In accordance with certain examples, the methods disclosed herein may be implemented using, at least in part, a computer system. The computer systems may be, for example, general-purpose computers such as those based on Unix, Intel PENTIUM-type processor, Motorola PowerPC, Sun UltraSPARC, Hewlett-Packard PA-RISC processors, or any other type of processor. It should be appreciated that one or more of any type computer system may be used according to various embodiments of the technology. Further, the system may be located on a single computer or may be distributed among a plurality of computers attached by a communications network. A general-purpose computer system according to one embodiment may be configured to perform any of the described functions including but not limited to: data acquisition, data analysis, peak finding, peak fitting, peak area determination, peak intensity determination, peptide sequence identification, protein sequence determination and the like. It should be appreciated that the system may perform other functions, including network communication, and the technology is not limited to having any particular function or set of functions.

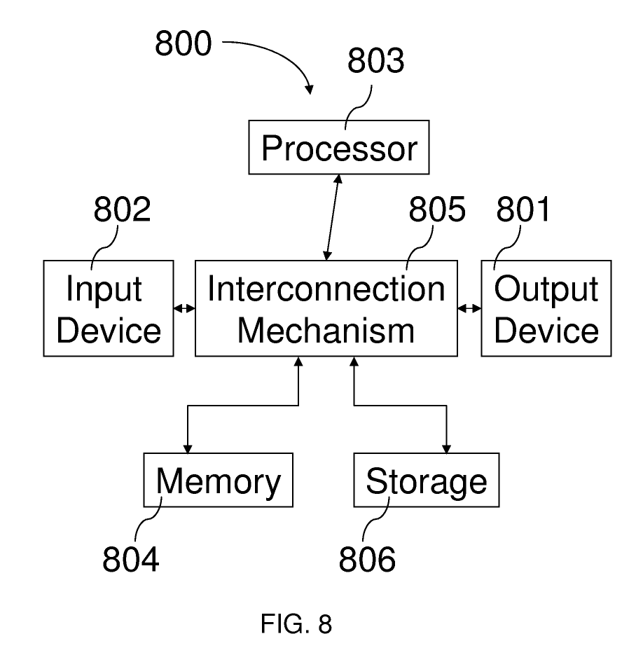

For example, various aspects may be implemented as specialized software executing in a general-purpose computer system 800 such as that shown in FIG. 8. The computer system 800 may include a processor 803 connected to one or more memory devices 804, such as a disk drive, memory, or other device for storing data. Memory 804 is typically used for storing programs and data during operation of the computer system 800. Components of computer system 800 may be coupled by an interconnection mechanism 805, which may include one or more busses (e.g., between components that are integrated within a same machine) and/or a network (e.g., between components that reside on separate discrete machines). The interconnection mechanism 805 enables communications (e.g., data, instructions) to be exchanged between system components of system 800. The computer system 800 typically is electrically coupled to an interface on a MS/MS such that electrical signals may be provided from the MS/MS to the computer system 800 for storage and/or processing.

Computer system 800 may also include one or more input devices 802, for example, a keyboard, mouse, trackball, microphone, touch screen, and one or more output devices 801, for example, a printing device, display screen, speaker. In addition, computer system 800 may contain one or more interfaces (not shown) that connect computer system 800 to a communication network (in addition or as an alternative to the interconnection mechanism 805.

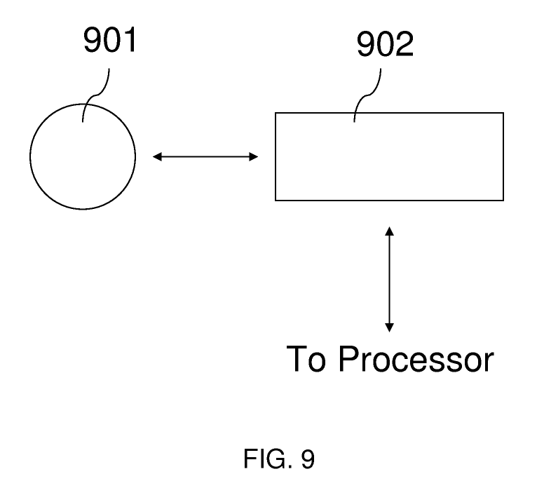

The storage system 806, shown in greater detail in FIG. 9, typically includes a computer readable and writeable nonvolatile recording medium 901 in which signals are stored that define a program to be executed by the processor or information stored on or in the medium 901 to be processed by the program. For example, the peak finding algorithm used in certain embodiments disclosed herein may be stored on the medium 901. The medium may, for example, be a disk or flash memory. Typically, in operation, the processor causes data to be read from the nonvolatile recording medium 901 into another memory 902 that allows for faster access to the information by the processor than does the medium 901. This memory 902 is typically a volatile, random access memory such as a dynamic random access memory (DRAM) or static memory (SRAM). It may be located in storage system 806, as shown, or in memory system 804. The processor 803 generally manipulates the data within the integrated circuit memory 804, 902 and then copies the data to the medium 901 after processing is completed. A variety of mechanisms are known for managing data movement between the medium 901 and the integrated circuit memory element 804, 902, and the technology is not limited thereto. The technology is not limited to a particular memory system 804 or storage system 806.

The computer system may also include specially-programmed, special-purpose hardware, for example, an application-specific integrated circuit (ASIC). Aspects of the technology may be implemented in software, hardware or firmware, or any combination thereof. Further, such methods, acts, systems, system elements and components thereof may be implemented as part of the computer system described above or as an independent component.

Although computer system 800 is shown by way of example as one type of computer system upon which various aspects of the technology may be practiced, it should be appreciated that aspects are not limited to being implemented on the computer system as shown in FIG. 8. Various aspects may be practiced on one or more computers having a different architecture or components than that shown in FIG. 8. Computer system 800 may be a general-purpose computer system that is programmable using a high-level computer programming language. Computer system 800 may be also implemented using specially programmed, special purpose hardware. In computer system 800, processor 803 is typically a commercially available processor such as the well-known Pentium class processor available from the Intel Corporation. Many other processors are available. Such a processor usually executes an operating system which may be, for example, the Windows 95, Windows 98, Windows NT, Windows 2000 (Windows ME), Windows XP or Windows Vista operating systems available from the Microsoft Corporation, MAC OS System X operating system available from Apple Computer, the Solaris operating system available from Sun Microsystems, or UNIX or Linux operating systems available from various sources. Many other operating systems may be used.

The processor and operating system together define a computer platform for which application programs in high-level programming languages are written. It should be understood that the technology is not limited to a particular computer system platform, processor, operating system, or network. Also, it should be apparent to those skilled in the art that the present technology is not limited to a specific programming language or computer system. Further, it should be appreciated that other appropriate programming languages and other appropriate computer systems could also be used.

In certain examples, the hardware of software is configured to implement cognitive architecture, neural networks or other suitable implementations. For example, a protein sequence database may be linked to the system to provide access to existing protein sequence data or results. Such configuration would allow for storage and access of large known protein sequences, which can increase the accuracy of the methods disclosed herein.

One or more portions of the computer system may be distributed across one or more computer systems coupled to a communications network. These computer systems also may be general-purpose computer systems. For example, various aspects may be distributed among one or more computer systems configured to provide a service (e.g., servers) to one or more client computers, or to perform an overall task as part of a distributed system. For example, various aspects may be performed on a client-server or multi-tier system that includes components distributed among one or more server systems that perform various functions according to various embodiments. These components may be executable, intermediate (e.g., IL) or interpreted (e.g., Java) code which communicate over a communication network (e.g., the Internet) using a communication protocol (e.g., TCP/IP). It should also be appreciated that the technology is not limited to executing on any particular system or group of systems. Also, it should be appreciated that the technology is not limited to any particular distributed architecture, network, or communication protocol.

Various embodiments may be programmed using an object-oriented programming language, such as SmallTalk, Basic, Java, C++, Ada, or C# (C-Sharp). Other object-oriented programming languages may also be used. Alternatively, functional, scripting, and/or logical programming languages may be used. Various aspects may be implemented in a non-programmed environment (e.g., documents created in HTML, XML or other format that, when viewed in a window of a browser program, render aspects of a graphical-user interface (GUI) or perform other functions). Various aspects may be implemented as programmed or non-programmed elements, or any combination thereof.

In certain examples, a user interface may be provided such that a user may enter protein sequences, isobaric labels and the like for use by the computer system in quantifying the peptides. For example, in instances where a user already knows the sequence of a peptide being quantified, the user can enter that sequence (or import the sequence from a text file) and enter the corresponding label used to label the peptide. In instances where a user does not know the sequence, the user may select a box indicating that the peptide sequence is unknown. The system may arbitrarily assign the peptide a name, e.g., unknown #1, so that the amount of label that is quantified may be correlated to some entry in a list. The user interface may also include fields for inputting user notes or the like. Other features for inclusion in a user interface will be readily selected by the person of ordinary skill in the art, given the benefit of this disclosure.

In accordance with certain examples, the methods disclosed herein may be used in clinical diagnostics. For example, one or more biomarkers or analytes may be analyzed and quantified using the methods disclosed herein. In particular, a plurality of different patient samples may be analyzed using the methods disclosed herein. For example, it may be possible to combine urine samples from several dozen patients by labeling each patient sample with one or more unique isobaric labels, combining the samples and then analyzing them for a particular peptide or protein of interest. It will be within the ability of the person of ordinary skill in the art, given the benefit of this disclosure to perform clinical measurements of patient samples using the methods disclosed herein

In accordance with certain examples, the methods disclosed herein may be used to determine the cellular response to a particular drug or therapeutic. Protein levels in control subjects and subjects exposed to a particular drug or therapeutic may be monitored to determine whether or not a particular drug or therapeutic causes an increase or decrease in protein expression. The rapid analysis provided by the methods disclosed herein may permit real-time monitoring of protein levels as a drug or therapeutic becomes active and then is removed or excreted through one or more metabolic processes. It will be within the ability of the person of ordinary skill in the art, given the benefit of this disclosure, to identify particular cellular responses to a drug or therapeutic using the methods disclosed herein.

In accordance with certain examples, the methods disclosed herein may be used to monitor the response of a plant to an agricultural product. For example, a plant may be exposed to an agricultural product, and as a result, the plant may produce increased levels of a protein which may, for example, provide increased drought-resistance, insect-resistance, disease-resistance or the like. Increased or decreased protein levels may be monitored in response to the agricultural product. The methods disclosed herein may be used to quantify the increase and/or decrease in protein expression after such exposure. The person of ordinary skill in the art, given the benefit of this disclosure, will be able to use the methods disclosed herein in an agricultural setting.

In accordance with certain examples, the methods disclosed herein may be used in mode of action experiments. In such experiments, a subject, such as a mammal, is administered an agent or environmental stress and the changes in phenotype may be monitored. In particular, after administration of an agent, the level of expression of a protein many be monitored to ascertain if the agent caused the protein level to change. Such changes may then be correlated with an observed phenotypical change.

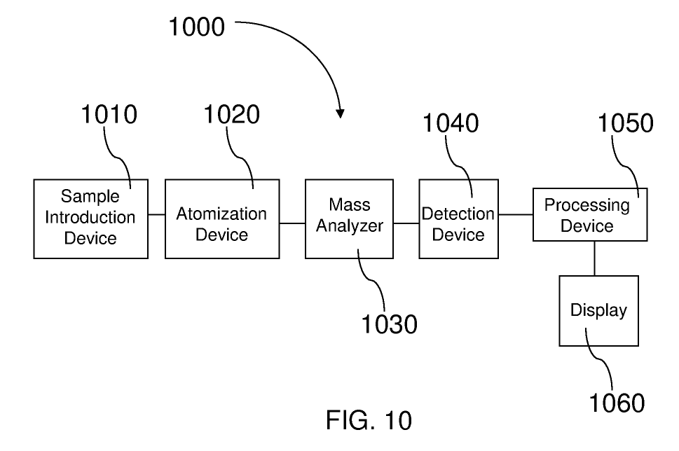

In accordance with certain examples, a mass spectrometer configured to quantify two or more peptides is disclosed. An illustrative device for mass spectroscopy (MS) is schematically shown in FIG. 10. An MS device 1000 includes a sample introduction device 1010 in fluid communication with an atomization device 1020, e.g., a plasma, a mass analyzer 1030, a detection device 1040, a processing device 1050 and a display 1060. The sample introduction device 1010, the atomization device 1020, the mass analyzer 1030 and the detection device 1040 may be operated at reduced pressures using one or more vacuum pumps. In certain examples, however, only one or more of the mass analyzer 1030 and/or the detection device 1040 are operated at reduced pressures. The sample introduction device 1010 may include an inlet system configured to provide sample to the atomization device 1020. The inlet system may include one or more batch inlets, direct probe inlets and/or chromatographic inlets. The sample introduction device 1010 may be an injector, a nebulizer or other suitable devices that can deliver solid, liquid or gaseous samples to the atomization device 1020. The mass analyzer 1030 can take numerous forms depending generally on the sample nature, desired resolution, etc. and exemplary mass analyzers are discussed further below. The detection device 1040 can be any suitable detection device that can be used with existing mass spectrometers, e.g., electron multipliers, Faraday cups, coated photographic plates, scintillation detectors, etc. and other suitable devices that will be selected by the person of ordinary skill in the art, given the benefit of this disclosure. The processing device 1050 typically includes a microprocessor and/or computer and suitable software for analysis of samples introduced into the MS device 1000. One or more databases can be accessed by the processing device 1050 for determination of the identity of species introduced into the MS device 1000. Other suitable additional devices known in the art can also be used with the MS device 1000 including, but not limited to, autosamplers, such as AS-90plus and AS-93plus autosamplers commercially available from PerkinElmer, Inc.

In accordance with certain examples, the mass analyzer of MS device 1000 can take numerous forms depending on the desired resolution and the nature of the introduced sample. In certain examples, the mass analyzer is a scanning mass analyzer, a magnetic sector analyzer (e.g., for use in single and double-focusing MS devices), a quadrupole mass analyzer, an ion trap analyzer (e.g., cyclotrons, quadrupole ions traps), time-of-flight analyzers (e.g., matrix-assisted laser desorbed ionization time of flight analyzers), and other suitable mass analyzers that can separate species with different mass-to-charge ratios. The methods disclosed herein may be used with any one or more of the mass analyzers listed above and other suitable mass analyzers.

In accordance with certain other examples, the mass spectrometer may be configured with one or more atomization/ionization devices. For example, an electron impact source, a chemical ionization source, a field ionization source, desorption sources such as, for example, those sources configured for fast atom bombardment, field desorption, laser desorption, plasma desorption, thermal desorption, electrohydrodynamic ionization/desorption, etc., thermospray or electrospray ionization sources may be used. It will be within the ability of the person of ordinary skill in the art, given the benefit of this disclosure, to select suitable devices for atomization/ionization for the MS devices disclosed herein.

In accordance with certain other examples, the MS device disclosed here can be hyphenated with one or more other analytical techniques. When such devices are hyphenated, the two devices may be placed in fluid communication. As used herein “fluid communication” refers to coupling at least some part of the two devices such that a fluid, e.g., a gas, a liquid or other state that can flow, may pass from one of the devices to the other device. For example, the MS device can be hyphenated with devices for performing liquid chromatography, gas chromatography, capillary electrophoresis, and other suitable separation techniques. When coupling an MS device to another analytical technique, it may be desirable to include a suitable interface, e.g., traps, jet separators, etc., to introduce sample into the MS device from the other device. When coupling an MS device to a liquid chromatograph, it may also be desirable to include a suitable interface to account for the differences in volume used in liquid chromatography and mass spectroscopy. For example, split interfaces can be used so that only a small amount of sample exiting the liquid chromatograph is introduced into the MS device. Sample exiting from the liquid chromatograph may also be deposited in suitable wires, cups or chambers for transport to the low flow plasma of the MS device. In certain examples, the liquid chromatograph includes a thermospray configured to vaporize and aerosolize sample as it passes through a heated capillary tube. Other suitable devices for introducing liquid samples from a liquid chromatograph into a MS device, or other detection device, will be readily selected by the person of ordinary skill in the art, given the benefit of this disclosure.

In certain examples, an MS device is hyphenated with at least one other MS device for tandem mass spectroscopy analyses. For example, one MS device can include a first type of mass analyzer and the second MS device can include a different or similar mass analyzer as the first MS device. In other examples, the first MS device may be operative to isolate the molecular ions and the second MS device may be operative to fragment/detect the isolated molecular ions. In certain embodiments, the first MS device may take any configuration provided that it can pass species having a selected mass-to-charge ratio to the second MS device. The second MS device may also take any form provided that it can fragment the isobaric label of the labeled analyte to provide a detectable signal. It will be within the ability of the person of ordinary skill in the art, given the benefit of this disclosure, to select or design suitable MS/MS devices for use with the methods disclosed herein.

In accordance with certain examples, a computer readable medium comprising an algorithm or program configured to quantify simultaneously two or more peptides is disclosed. In certain examples, the computer readable medium may be a floppy disk, a compact disc, DVD media, Blu-Ray media, HD-DVD media, a hard disk, a USB drive or the like. Other suitable media will be readily selected by the person of ordinary skill in the art, given the benefit of this disclosure. In certain examples, a computer readable medium may include instruction or a link for downloading or accessing an algorithm from the Internet, a web-site, an ftp site or other comparable site that can include one or more links to the algorithm. The computer readable medium may optionally include instructions for using the algorithm or program to quantify two or more peptides.

In accordance with certain examples, the algorithm or software may be configured to operate on the processing device or computer of one or both MS devices. For example, either or both of the MS devices may be electrically coupled to a processing device that can use an algorithm to quantify isobaric label fragments and/or peptides.

In accordance with certain examples, a kit comprising an isobaric label and a computer readable medium is disclosed. In certain examples, the computer readable medium comprises an algorithm configured to quantify simultaneously at least two peptides, e.g., using the methods described herein. In some examples, the kit may further include instructions for using the isobaric label and/or the algorithm in the kit. In certain examples, the kit may include two or more isobaric labels.

Certain specific examples are provided in more detail below to illustrate further the novel technology described herein.

### EXAMPLE 1

Insulin samples from two different patients may be simultaneously analyzed using the methods disclosed herein. As an example of the analysis of insulin, two serum samples suspected of containing insulin may be analyzed. Since insulin contains six cysteine residues, it is readily labeled with isobaric mass tags containing a thiol-reactive functionality, such as iodoacetamide. Serum from a first patient may be labeled with a first iodoacretamide functionalized isobaric label. Serum from a second patient may be labeled with a second iodoacretamide functionalized isobaric label. The two labeled serum samples may be proteolytically or chemically cleaved, combined together and injected into a tandem MS/MS spectrometer. The first MS device may be configured to select an isobaric mass tag-labeled insulin species from the rest of the sample. The second MS device may be configured to fragment the two labeled insulin species such that a low mass fragment from each of the first label and the second label results. The first and second labels may be selected such that the low mass fragment of the two labels differs by a selected mass-to-charge ratio, e.g., differ by 3 atomic mass units. Fragmentation of the labels permits distinction both insulin samples even though the insulin-containing samples originate from different patients. The relative amount of insulin in each sample may then be determined by applying a mathematical function to the resulting spectra to provide a fitted curve. The areas under the peaks of the fitted curve may be used to determine the relative amounts of insulin present. Alternatively, a third label may be used with a known amount of insulin to provide an internal standard to allow for determination of the absolute amount of insulin present in the two patient samples.

### EXAMPLE 2

The intensity of each low mass fragment may be determined to quantify the amount of peptide present in a sample. In an illustrative embodiment, the following seven different isobaric labels may be used. Bold and underlining means the residue is an isotopically heavy version of an amino acid.

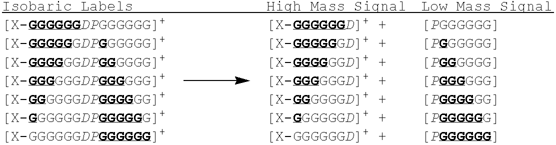

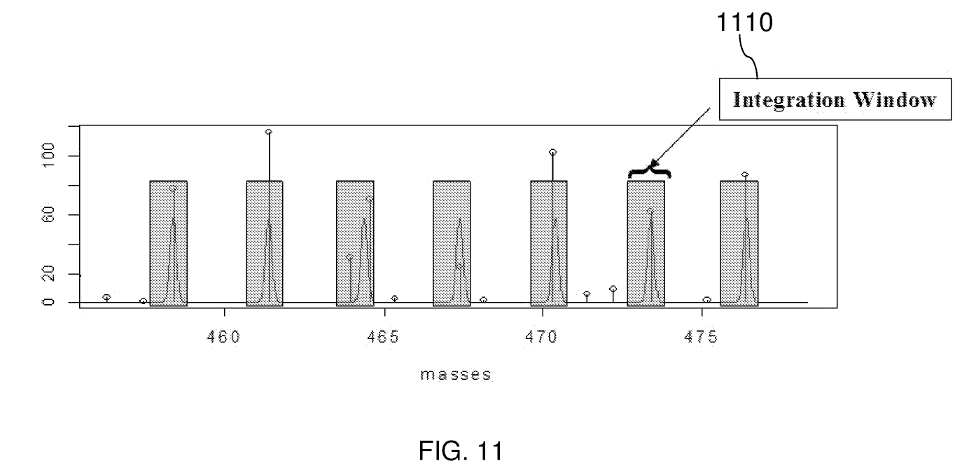

Analysis of the fragments providing the low mass signal will provide about seven peaks, one for each label, separated by about 3 atomic mass units. FIG. 11 shows a graph of relative intensity versus mass-to-charge. As can be seen in FIG. 11, there are 7 large groups of peaks with each peak representing one of the labels listed above. For illustration, the third group from the left includes two peaks which may result from a processing error by the instrument. The methods disclosed herein may be designed to account for such processing errors by grouping peaks close together and by accounting for the known mass-to-charge ratios of the label fragments, e.g., 3 amu separation of the fragments. Also shown in FIG. 11 is an integration window 1110. The integration window 1110 runs from baseline to baseline around a peak to provide a window large enough to account for the area and/or intensity of each peak. The area under each of the curves, or the intensity of each of the curves, may be approximated by fitting a mathematical function to all of the peaks.

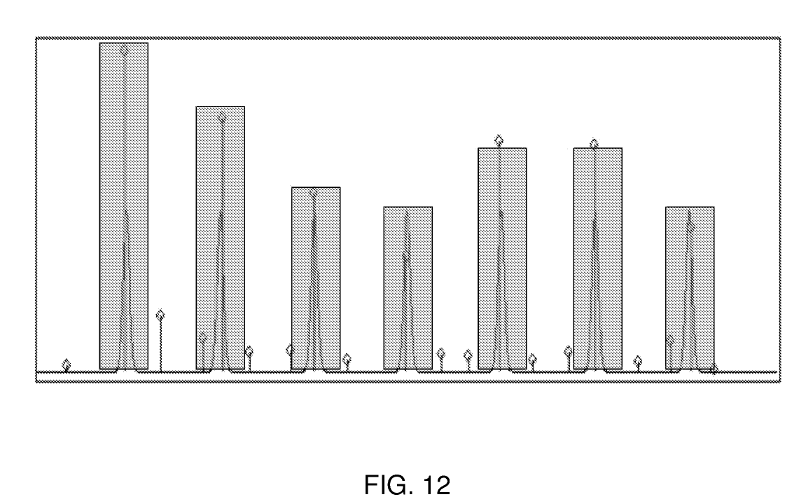

Referring now to FIG. 12, the intensity of each peak may be used to determine the relative amount of each peak. The relative amount may be determined by summing the intensities and then dividing the intensity for any particular peak by the summed intensities. This process will provide a relative amount of each label, and correspondingly, a relative amount of each peptide, present in a sample.

### EXAMPLE 3

The following example describes one way that may be implemented in software to find the peaks within a mass spectrum or a selected mass-to-charge window in a mass spectrum.

First, a peak list with N mass peak and intensity pairs (mᵢ, Iᵢ) is used. In that set of peaks, there should be a set of peaks resulting from isobaric labels at the masses Mⱼ. The fit function is defined as F(m)=ΣPⱼ(m) where Pⱼ(m) is the function used to fit each expected mass peak for the j-th isobaric label individually. For this example, a Gaussian function is used to fit each individual label.

The trial fit is defined as TF(δ)=Σₗ IₗF(mᵢ+δ). δ is a shift in the mass scale used to find an optimal fit. To find an optimal fit, a series of trial fits may be made and the mass shift of the trial fit with the maximum result is selected as the best-fit (δ_{best}).

Once the best fit is found, the intensities for the isobaric labels fragments of interest may be extracted by adding the intensity of all mass peaks found within a mass window (w) around the expected isobaric label fragment peaks offset by the best fit mass delta (Mⱼ+δ_{best}) found during the fitting process.

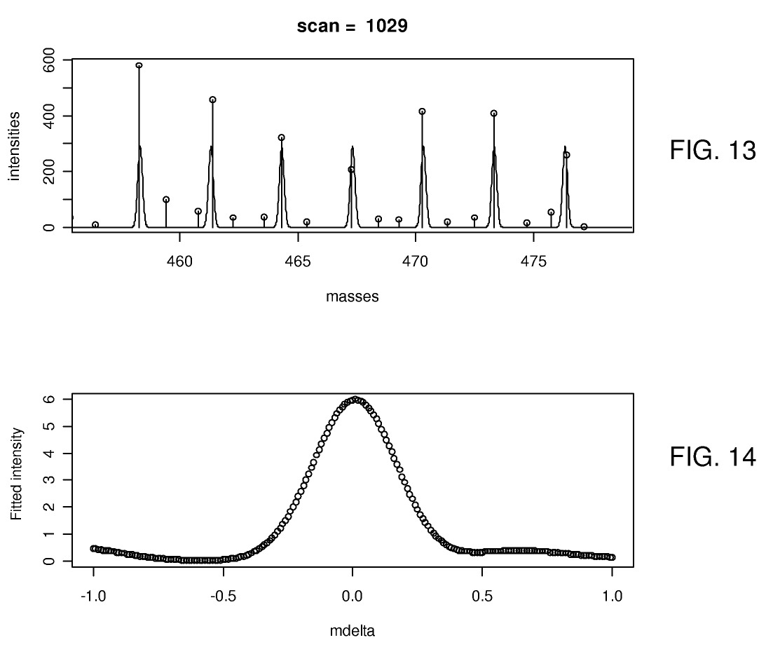

Referring to FIG. 13, mass peaks are shown as the lines with circles at the top. A trial fit function is shown as the continuous a line. The best fit is shown. The mass scale is in atomic mass units (AMU's) and the intensities are in counts. Referring to FIG. 14, the fit values obtained for a series of mass deltas ranging from −1 to 1 AMU are shown. The best fit is the maximum of this function. Is the curve shown in FIG. 14, the best fit is near a mass delta of zero.

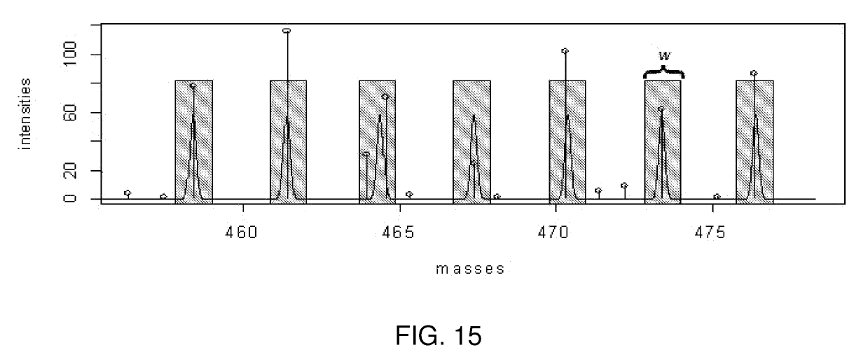

After fitting the function to the peaks, the intensities around the adjusted isobaric label masses may be added to extract the intensity for that label as shown by the shaded boxes in FIG. 15.

### EXAMPLE 4

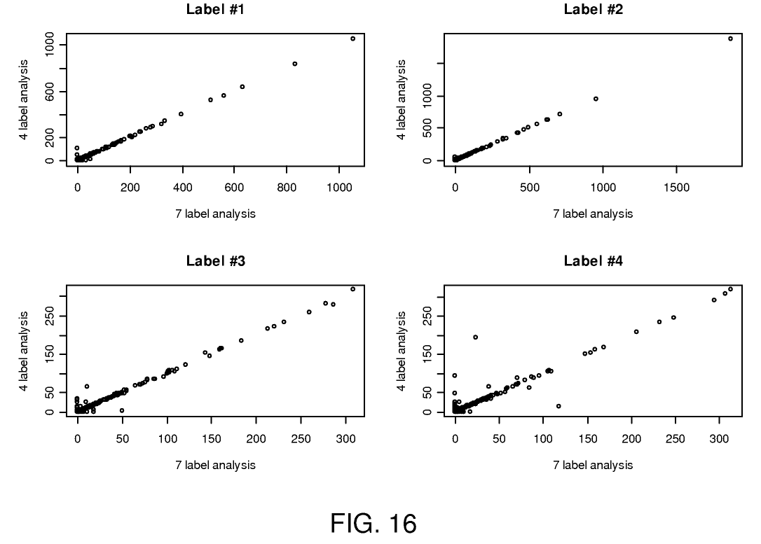

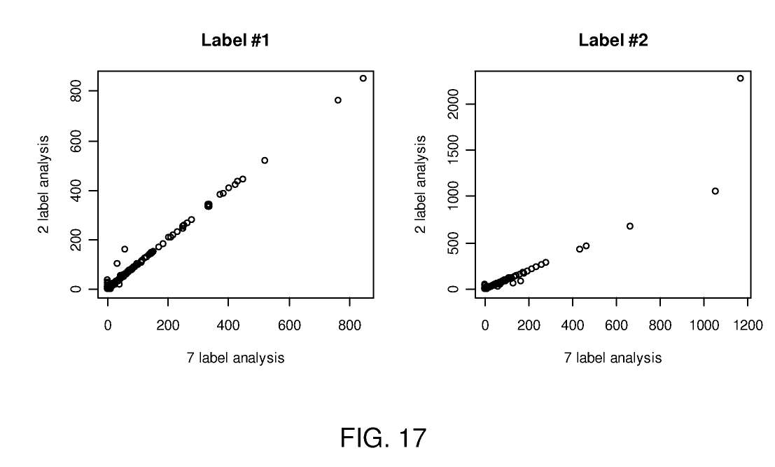

To determine the effectiveness of the peak finding algorithm described in Example 3 on 2 or 4 label samples, a single data set that contains 7 labels was analyzed by picking 7, 4 or 2 label peaks. The results from experiments with seven picked peaks were compared with the results from experiments picking 2 or 4 peaks (see FIGS. 16 and 17). In all cases, there was almost a 1:1 correspondence, which indicates the algorithm may be used to pick 2, 4, 7 or more peaks with good accuracy.

When introducing elements of the examples disclosed herein, the articles “a,” “an,” and “the” are intended to mean that there are one or more of the elements. The terms “comprising,” “including” and “having” are intended to be open ended and mean that there may be additional elements other than the listed elements. It will be recognized by the person of ordinary skill in the art, given the benefit of this disclosure, that various components of the examples can be interchanged or substituted with various components in other examples.

Although certain features, aspects, examples and embodiments have been described above, additions, substitutions, modifications, and alterations of the disclosed illustrative features, aspects, examples and embodiments will be readily recognized by the person of ordinary skill in the art, given the benefit of this disclosure.

What is claimed is:

1. A method of quantifying two or more isobaric labels comprising:
applying a mathematical function to at least two isobaric label fragment peaks in a mass spectrum to provide a fitted curve; and
determining a relative amount of each isobaric label from the fitted curve.

2. The method of claim 1 further comprising applying the mathematical function to at least two low mass, isobaric label fragment peaks in the mass spectrum.

3. The method of claim 1 further comprising applying the mathematical function to at least two high mass, isobaric label fragment peaks in the mass spectrum.

4. The method of claim 1 further comprising applying the mathematical function to all isobaric label fragment peaks in the mass spectrum.

5. The method of claim 1 wherein each isobaric label fragment peak has an intensity and in which the determining step further comprises:
determining the intensity of the at least two isobaric label fragment peaks in the mass spectrum from the fitted curve;
summing the intensity of the at least two isobaric label fragment peaks in the mass spectrum; and
dividing the intensity of at least one isobaric label fragment peak by the summed intensity to determine a relative amount of the at least one isobaric label fragment peak present in a sample.

6. The method of claim 1 in which the determining step further comprises:
determining an area for each of the at least two isobaric label fragment peaks in the mass spectrum from the fitted curve;
summing the areas of the at least two isobaric label fragment peaks in the mass spectrum; and
dividing the area of at least one isobaric label fragment peak by the summed areas to determine the relative amount of the at least one isobaric label fragment peak.

7. The method of claim 1 in which the applying a mathematical function comprises fitting the mathematical function to each of the at least two isobaric label fragment peaks in the mass spectrum to provide the fitted curve.

8. The method of claim 7 in which the mathematical function comprises at least one Gaussian function.

9. A method of quantifying peptides comprising:
subjecting a sample to fragmentation in a mass spectrometer, the sample comprising a first peptide labeled with a first isobaric label and a second peptide labeled with a second isobaric label, in which fragmentation in the mass spectrometer provides a low mass fragment peak and a high mass fragment peak for each of the first and second isobaric labels;
applying a mathematical function to at least one of the low mass fragment peaks or the high mass fragment peaks to provide a fitted curve; and
determining a relative amount of each of the first peptide and the second peptide from the fitted curve.

10. The method of claim 9 further comprising configuring the first isobaric label and the second isobaric label to provide low mass fragments having different mass-to-charge ratios.

11. The method of claim 10 further comprising configuring the mass-to-charge ratios to differ by at least 3 atomic mass units.

12. The method of claim 9 wherein each low mass fragment peak and each high mass fragment peak has an intensity, and in which the determining step further comprises:
determining the intensity of the peaks of the fitted curve;
summing the intensity of the peaks of the fitted curve;
dividing the intensity of at least one of the fitted curve peaks by the summed intensity to determine a relative amount of at least one isobaric label fragment peak; and
correlating the determined relative amount of the at least one isobaric label fragment peak with a relative amount of a peptide.

13. The method of claim 9 in which the first peptide and the second peptide have the same composition but are derived from at least two different samples.

14. The method of claim 9 in which the first peptide and the second peptide have different compositions or sequences.

15. A mass spectrometer comprising:
a first stage configured to select at least two peptides labeled with two, different isobaric labels;
a second stage in fluid communication with the first stage, the second stage configured to fragment the two, different isobaric labels of the selected at least two peptides to provide two low mass fragments and two high mass fragments;
a processing device electrically coupled to the second stage, the processing device configured to quantify simultaneously the two or more peptides.

16. The mass spectrometer of claim 15 in which each of the first stage and the second stage is configured as a quadrupole mass spectrometer.

17. The mass spectrometer of claim 15 in which the first stage is configured to pass two or more peptides with a mass-to-charge ratio within a selected mass-to-charge window.

18. The mass spectrometer of claim 15 in which the processing device further comprises an algorithm that is configured to quantify the peptides by fitting a mathematical function to the two low mass fragments to quantify simultaneously a relative amount of the at least two peptides.

19. A kit comprising:
an isobaric label;
a computer readable medium comprising an algorithm configured to quantify simultaneously at least two peptides; and
instructions for using the isobaric label and the algorithm to quantify peptides in a sample.

20. The kit of claim 19 in which the kit further comprises two or more isobaric labels.

21. The kit of claim 19 in which the algorithm is configured to quantify the at least two peptides by fitting a mathematical function to two low mass fragments from isobaric labels to quantify simultaneously a relative amount of the at least two peptides.

22. A method of quantifying at least two peptides labeled with different isobaric labels, the method comprising fitting a mathematical function to two isobaric label fragment peaks in a mass spectrum to quantify simultaneously a relative amount of the at least two peptides present in a sample.

23. A computer readable medium comprising an algorithm for simultaneously quantifying at least two peptides, the algorithm configured to fit a mathematical function to at least two isobaric label fragment peaks in a mass spectrum to quantify simultaneously a relative amount of the at least two peptides.
